(19) United States
(12) Patent Application Publication
(10) Pub. No.: US 2022/0023249 A1
(43) Pub. Date: Jan. 27, 2022

(54) Carotenoid Formulation For Increased Bioavailability

(71) Applicant: Industrial Organica, SA de CV, Monterrey (MX)

(72) Inventors: John Nolan, Waterford (IE); Alfonso Prado-Cabrero, Six Cross Roads (IE); Marina Green, Waterford (IE); Jose Torres Quiroga, Monterrey (MX); Carlos Torres Gomez, Monterrey (MX); Jazmin Marquez Santacruz, General Escobedo (MX)

(73) Assignee: Industrial Organica, SA de CV, Monterrey (MX)

(21) Appl. No.: 17/377,092

(22) Filed: Jul. 15, 2021

Related U.S. Application Data
(60) Provisional application No. 63/054,653, filed on Jul. 21, 2020.

(51) Int. Cl. A61K 31/235; A61K 31/047; A61K 47/24; A61K 33/30; A61K 33/34; A61K 9/48; A61K 36/28; A61K 47/22

(52) U.S. Cl. CPC A61K 31/235; A61K 31/047; A61K 47/24; A61K 47/22; A61K 33/34; A61K 9/4858; A61K 36/28; A61K 33/30

(57) ABSTRACT

A composition including a xanthophyll carotenoid diacetate, a transition metal salt, and phospholipids is provided. The composition does not include micelles and is not an emulsion. Methods of supporting eye health in subjects in need thereof using the composition are also provided.

### CROSS-REFERENCE TO RELATED APPLICATIONS

This application claims the benefit of U.S. Provisional Application No. 63/054,653, filed on Jul. 21, 2020. The entire disclosure of the above application is incorporated herein by reference.

### FIELD

The present disclosure relates to increasing the bioavailability of carotenoids.

### BACKGROUND

This section provides background information related to the present disclosure which is not necessarily prior art.

The retina is a tissue layer that includes light sensitive neurons. The retina is located at the back of the eye where light is focused into an image. By way of the optic nerve, the image is transmitted to the brain, where visual perception is created.

The macula is an area at the center of the retina that has a high concentration of photoreceptor cells known as cones. The macula supports central vision, most color vision, and fine details of what is seen.

The macula has a yellow pigment provided by xanthophyll carotenoids. The xanthophyll carotenoids include (3R,3′R,6R)-lutein, (3R,3′R)-zeaxanthin, and meso-zeaxanthin. The pigment absorbs blue light, thus protecting the macula from oxidative injury. When the pigment breaks down or deteriorates, the macula is subject to increased oxidative damage leading to the destruction of sharp central vision.

Macular degeneration, or age-related macular degeneration (AMD), is a chronic progressive eye disease characterized by the degeneration of the macula, which results in a loss of central vision. This disease is the leading cause of acquired legal blindness and visual impairment among people over the age of 50 in North America and in other societies. Because the pigment protects the macula by filtering short wavelength blue light and has antioxidant and optical properties, AMD can be treated by supplementing the pigment with xanthophyll carotenoids. Enrichment of macular pigment has been shown to enhance visual function for patients with AMD and individuals free of retinal pathology. However, some xanthophyll carotenoids are excreted at high levels, leaving little to be absorbed in the serum and delivered to the macula. Accordingly, it is desirable to increase the bioavailability of xanthophyll carotenoids.

### SUMMARY

This section provides a general summary of the disclosure, and is not a comprehensive disclosure of its full scope or all of its features.

In various aspects, the current technology provides a composition including a xanthophyll carotenoid diacetate, a transition metal salt, and phospholipids, wherein the composition does not include micelles and the composition is not an emulsion.

In one aspect, the phospholipids are selected from the group consisting of phosphatidylcholines, lysophosphatidylcholines, phosphatidic acids, phosphatidylethanolamines, phosphatidylglycerols, phosphatidylserines, phosphoinositides, phosphosphingolipids, and combinations thereof.

In one aspect, the xanthophyll carotenoid diacetate includes meso-zeaxanthin diacetate.

In one aspect, the composition also includes (3R,3′R)-zeaxanthin diacetate, (3R,3′R,6R)-lutein diacetate, or a combination thereof.

In one aspect, the composition also includes (3R,3′R,6R)-lutein, (3R,3′R)-zeaxanthin, meso-zeaxanthin, esters thereof, diacetates thereof, and combinations thereof.

In one aspect, the composition is configured such that micelles encapsulating the xanthophyll carotenoid in free form are formed in a digestive tract of a subject after the composition is orally administered to the subject.

In one aspect, the composition also includes an antioxidant.

In one aspect, the transition metal salt includes zinc oxide, cupric oxide, cuprous oxide, or combinations thereof.

In one aspect, the composition is provided in a soft gel capsule.

In various aspects, the current technology also provides a method of supporting good eye health in a subject in need thereof, the method including administering a safe and effective amount of a carotenoid composition to the subject, the carotenoid composition including a xanthophyll carotenoid diacetate, a transition metal salt, and phospholipids, wherein the carotenoid composition does not include micelles, the composition is not an emulsion, and micelles encapsulating the xanthophyll carotenoid in free form are formed from the phospholipids within the digestive tract of the subject.

In one aspect, the subject is a human or non-human mammal having below normal levels of macular pigments or at risk of developing AMD.

In one aspect, the xanthophyll carotenoid diacetate is meso-zeaxanthin diacetate and the carotenoid composition is a gel capsule including greater than or equal to about 1% (w/w) to less than or equal to about 30% (w/w) of the meso-zeaxanthin diacetate.

In one aspect, the carotenoid composition further includes (3R,3′R,6R)-lutein and (3R,3′R)-zeaxanthin, the (3R,3′R,6R)-lutein and (3R,3′R)-zeaxanthin optionally being in diacetate forms, and the meso-zeaxanthin diacetate and (3R,3′R,6R)-lutein are provided in a meso-zeaxanthin diacetate:(3R,3′R,6R)-lutein ratio of from about 1:10 to about 10:1 and the meso-zeaxanthin diacetate and the (3R,3′R)-zeaxanthin are provided in a meso-zeaxanthin diacetate:(3R,3′R)-zeaxanthin ratio of from about 1:1 to about 20:1.

In one aspect, the carotenoid composition includes the meso-zeaxanthin diacetate, (3R,3′R,6R)-lutein, and (3R,3′R)-zeaxanthin in a meso-zeaxanthin diacetate:(3R,3′R,6R)-lutein:(3R,3′R)-zeaxanthin ratio of about 10:10:2.

In one aspect, the transition metal salt includes at least one of zinc or copper and the carotenoid composition further includes sunflower seed oil and at least one of vitamin C or vitamin E.

In various aspects, the current technology further provides a method of improving the bioavailability of meso-zeaxanthin in a subject, the method including converting meso-zeaxanthin diacetate into meso-zeaxanthin in free form in the digestive tract of the subject and forming micelles within the digestive tract of the subject, the micelles including a monolayer of phospholipids encapsulating the meso-zeaxanthin in free form, wherein more of the meso-zeaxanthin in free form remains biologically available within the blood stream of the subject than in corresponding meso-zeaxanthin when administered to the subject in crystalline form.

In one aspect, the forming micelles within the digestive tract of the subject is a result of administering a safe and effective amount of a carotenoid composition to the subject, the carotenoid composition including the phospholipids, the meso-zeaxanthin diacetate, and a transition metal salt, wherein the carotenoid composition is not an emulsion and does not include micelles when administered.

In one aspect, the carotenoid composition does not include gluten.

In one aspect, the carotenoid composition further includes meso-zeaxanthin, (3R,3′R)-zeaxanthin diacetate, (3R,3′R)-zeaxanthin, (3R,3′R,6R)-lutein diacetate, (3R,3′R,6R)-lutein, or combinations thereof.

In one aspect, the subject is a human or non-human mammal desiring to maintain or improve macular pigment levels.

In one aspect, the subject is a human or non-human mammal having or at risk of developing AMD.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIGS. 1A-1C show structures of the ester form of meso-zeaxanthin (FIG. 1A), meso-zeaxanthin in free form (FIG. 1B), and meso-zeaxanthin diacetate in accordance with various aspects of the current technology (FIG. 1C).

FIGS. 2A-2C show structures of the ester form of (3R,3′R)-zeaxanthin (FIG. 2A), (3R,3′R)-zeaxanthin in free form (FIG. 2B), and (3R,3′R)-zeaxanthin diacetate in accordance with various aspects of the current technology (FIG. 2C).

FIGS. 3A-3C show structures of the ester form of (3R,3′R,6R)-lutein (FIG. 3A), (3R,3′R,6R)-lutein in free form (FIG. 3B), and (3R,3′R,6R)-lutein diacetate in accordance with various aspects of the current technology (FIG. 3C).

FIGS. 4A-4C are schematic illustrations showing a partial metabolism of xanthophyll carotenoid compositions in accordance with various aspects of the current technology. FIG. 4A shows a stomach, FIG. 4B shows a duodenum of a small intestine, and FIG. 4C shows an enterocyte.

FIG. 5 shows lutein diacetate solubilizate and crystallized carotenoids in nutritional supplements. Formulations include (3R,3′R,6R)-lutein from marigold flower in microcrystals as a diacetate derivative. (3R,3′R,6R)-lutein is present esterified with fatty acids in the flower. To extract this carotenoid, it is de-esterified and purified by crystallization. To solubilize these crystals and facilitate absorption in the digestive system, (3R,3′R,6R)-lutein and other hydroxy carotenoids can be re-esterified with acetate or propionate upon crystallization and resuspended in the flower's lipid matrix and added surfactants to maintain solubility of the carotenoid at ambient conditions. Although (3R,3′R,6R)-lutein is shown in FIG. 5, meso-zeaxanthin and zeaxanthin are subject to the same pathway.

FIG. 6 is a flow chart illustrating the screening, randomization, and follow-up of study participants allocated (3R,3′R,6R)-lutein (L), meso-zeaxanthin (MZ), and (3R,3′R)-zeaxanthin (Z). A total of two participants discontinued the interventions due to adverse events related to gastrointestinal symptoms, bloating, and gastric discomfort when taken in a fasted state. Lack of follow-up was due to loss of contact.

FIGS. 7A-7C are graphs showing serum concentrations levels of (3R,3′R,6R)-lutein (L) (FIG. 7A), (3R,3′R)-zeaxanthin (Z) (FIG. 7B), and meso-zeaxanthin (MZ) (FIG. 7C) at baseline (0 months) and 6 months.

FIGS. 8A-8B are graphs showing the effect of different formulations on change of serum concentrations (Group 1=L_{10 mg}+MZ_{10 mg}+Z_{10 mg}; Group 2=L_{10 mg}+MZ_{10 mg}+Z_{10 mg} split dose; Group 3=L_{10 mg}+MZ_{10 mg}+Z_{10 mg}+Omegas; Group 4=L_{10 mg}+MZ_{10 mg}+Z_{10 mg} diacetates; Group 5=placebo). Between-group differences in change in (3R,3′R)-zeaxanthin (Z) serum concentration (FIG. 8A) and meso-zeaxanthin (MZ) serum concentration (FIG. 8B) are expressed as change from baseline and 6 months. Z and MZ serum response in Group 4 was significantly higher compared to the other active interventions and placebo (p<0.000 to p=0.019).

FIG. 9 is a graph showing macular pigment optical volume (MPOV) changes between 0 and 6 months. The MPOV response was significantly higher in Groups 1 and 4 compared to Group 5 (the placebo), with p=0.001 to p=0.039.

FIGS. 10A-10B are graphs showing the relationship between change in carotenoid serum concentration and change in tissue (response). Linear regression analyses of total carotenoid serum concentrations and carotenoid skin score (r=0.528, p<0.001) (FIG. 10A) and MPOV (r=0.408, p=0.001) (FIG. 10B) are shown. Interventions are as follows: Group 1, L (10 mg)+MZ (10 mg)+Z (2 mg) provided in one capsule; Group 2, L (10 mg)+MZ (10 mg)+Z (2 mg) provided in two capsules; Group 3, L (10 mg)+MZ (10 mg)+Z (2 mg) provided in DHA (430 mg) and EPA (90 mg) in two capsules; Group 4, L diacetates (10 mg)+MZ diacetates (10 mg)+Z diacetates (2 mg) provided in one capsule; or Group 5, placebo (sunflower oil).

FIG. 11 is graph showing skin carotenoid concentration change over time for Groups 1-5.

FIG. 12 is a graph showing the bioavailability of meso-zeaxanthin in various forms over a 6-month period.

FIG. 13 is a graph showing the bioavailability of (3R,3′R,6R)-lutein in various forms over a 6-month period.

### DETAILED DESCRIPTION

Example embodiments are provided so that this disclosure will be thorough, and will fully convey the scope to those who are skilled in the art. Numerous specific details are set forth such as examples of specific compositions, components, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, elements, compositions, steps, integers, operations, and/or components, but do not preclude the presence or addition of one or more other features, integers, steps, operations, elements, components, and/or groups thereof. Although the open-ended term “comprising,” is to be understood as a non-restrictive term used to describe and claim various embodiments set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, the present disclosure also specifically includes embodiments consisting of, or consisting essentially of, such recited compositions, materials, components, elements, features, integers, operations, and/or process steps. In the case of “consisting of,” the alternative embodiment excludes any additional compositions, materials, components, elements, features, integers, operations, and/or process steps, while in the case of “consisting essentially of,” any additional compositions, materials, components, elements, features, integers, operations, and/or process steps that materially affect the basic and novel characteristics are excluded from such an embodiment, but any compositions, materials, components, elements, features, integers, operations, and/or process steps that do not materially affect the basic and novel characteristics can be included in the embodiment.

Any method steps described herein are not to be construed as necessarily requiring their performance in the particular order discussed or illustrated, unless specifically identified as an order of performance. It is also to be understood that additional or alternative steps may be employed, unless otherwise indicated.

Although the terms first, second, third, etc. may be used herein to describe various steps, elements, components, regions, layers and/or sections, these steps, elements, components, regions, layers and/or sections should not be limited by these terms, unless otherwise indicated. These terms may be only used to distinguish one step, element, component, region, layer or section from another step, element, component, region, layer or section. Terms such as “first,” “second,” and other numerical terms when used herein do not imply a sequence or order unless clearly indicated by the context. Thus, a first step, element, component, region, layer or section discussed below could be termed a second step, element, component, region, layer or section without departing from the teachings of the example embodiments.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

In addition, disclosure of ranges includes disclosure of all values and further divided ranges within the entire range, including endpoints and sub-ranges given for the ranges. As referred to herein, ranges are, unless specified otherwise, inclusive of endpoints and include disclosure of all distinct values and further divided ranges within the entire range. Thus, for example, a range of “from A to B” or “from about A to about B” is inclusive of A and B.

Example embodiments will now be described more fully with reference to the accompanying drawings.

By supplementing xanthophyll carotenoids of the macula, eye health can be maintained and the degenerative effects of AMD can be slowed or minimized. However, in order to fully realize the positive effects of xanthophyll carotenoid supplementation, the xanthophyll carotenoids should be highly bioavailable, so that they can reach the macula and restore the pigment. Accordingly, the current technology provides carotenoid compositions that result in increased xanthophyll carotenoid bioavailability relative to known carotenoid compositions.

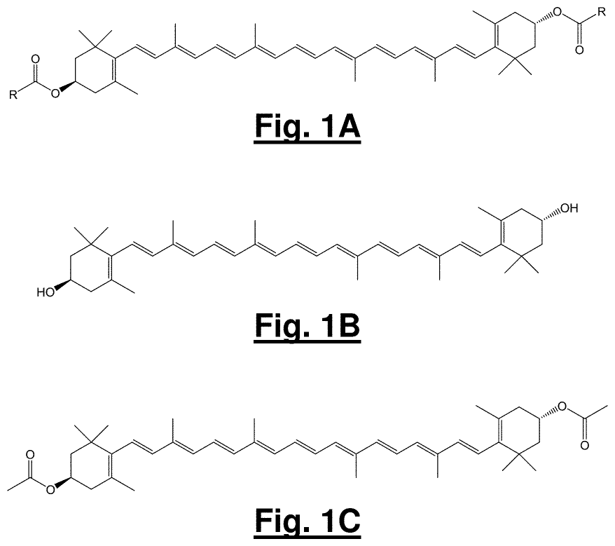

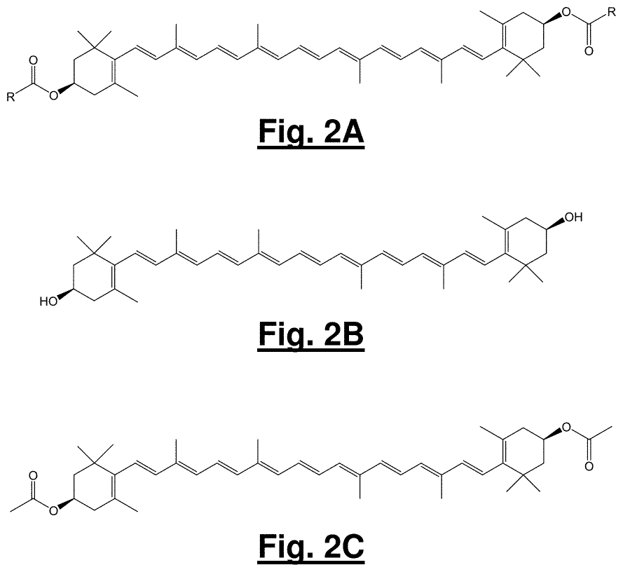

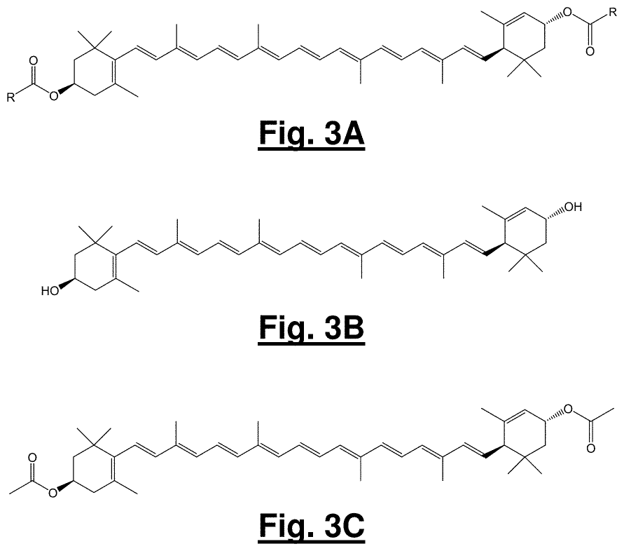

Xanthophyll carotenoids are found in extracts of various plants, such as from marigolds. As extracted, the xanthophyll carotenoids are in the form of esters. FIGS. 1A, 2A, and 3A show the xanthophyll carotenoids meso-zeaxanthin ester, (3R,3′R)-zeaxanthin ester, and (3R,3′R,6R)-lutein ester, respectively, where the R is an alkyl chain. In accordance with the current technology, the (3R,3′R)-zeaxanthin ester and (3R,3′R,6R)-lutein ester are subjected to saponification to form their respective structures in free form, as shown in FIGS. 2B and 3B, respectively. The meso-zeaxanthin in free form is obtained by a base-catalyzed isomerization of (3R,3′R,6R)-lutein. Then, the meso-zeaxanthin, (3R,3′R)-zeaxanthin, and (3R,3′R,6R)-lutein in free forms are acetylated to form meso-zeaxanthin diacetate, (3R,3′R)-zeaxanthin diacetate, and (3R,3′R,6R)-lutein diacetate as shown in FIGS. 1C, 2C, and 3C, respectively. The current technology provides compositions that include at least one of these xanthophyll carotenoid diacetates. The compositions also include a phospholipid.

In certain aspects, the current technology provides a carotenoid composition comprising a xanthophyll carotenoid diacetate and phospholipids. The xanthophyll carotenoid diacetate comprises meso-zeaxanthin diacetate (FIG. 1C), (3R,3′R)-zeaxanthin diacetate (FIG. 2C), (3R,3′R,6R)-lutein diacetate (FIG. 3C), or combinations thereof. In certain aspects, the xanthophyll carotenoid comprises meso-zeaxanthin diacetate and optionally further comprises at least one other xanthophyll carotenoid. When present, the at least one other xanthophyll carotenoid can be meso-zeaxanthin, a meso-zeaxanthin ester, (3R,3′R)-zeaxanthin diacetate, (3R,3′R)-zeaxanthin, a (3R,3′R)-zeaxanthin ester, (3R,3′R,6R)-lutein diacetate, (3R,3′R,6R)-lutein, a (3R,3′R,6R)-lutein ester, or combinations thereof, as non-limiting examples. It is understood that the xanthophyll carotenoid content and/or the carotenoid content of the composition can consist essentially of or consist of meso-zeaxanthin diacetate or meso-zeaxanthin diacetate together with any combination of the other exemplary carotenoids discussed herein. By “consists essentially of,” it is meant that the composition does not intentionally include additional carotenoids, including xanthophyll carotenoids; however, additional carotenoids may be unintentionally included as impurities, such as in concentrations of less than or equal to about 5% (w/w), less than or equal to about 2.5% (w/w), or less than or equal to about 1% (w/w) (based on the total weight of the carotenoid composition). It is understood that any exemplary composition described herein as comprising a xanthophyll carotenoid includes corresponding compositions that consist essentially of or consist of the xanthophyll carotenoid.

In various aspects, the carotenoid composition comprises at least one of meso-zeaxanthin, (3R,3′R)-zeaxanthin, or (3R,3′R,6R)-lutein, wherein the meso-zeaxanthin, (3R,3′R)-zeaxanthin, and (3R,3′R,6R)-lutein are individually and independently in base form, ester form, diacetate form, or combinations thereof, with the proviso that the composition comprises at least one of meso-zeaxanthin diacetate, (3R,3′R)-zeaxanthin diacetate, or (3R,3′R,6R)-lutein diacetate. When present, the meso-zeaxanthin and (3R,3′R,6R)-lutein (in diacetate forms, ester forms, diacetate forms, or combinations thereof) are provided in a meso-zeaxanthin:(3R,3′R,6R)-lutein ratio of from about 1:10 to about 10:1. When present, the meso-zeaxanthin and (3R,3′R)-zeaxanthin (in diacetate forms, ester forms, diacetate forms, or combinations thereof) are provided in a meso-zeaxanthin:(3R,3′R)-zeaxanthin ratio of from about 1:1 to about 20:1. As a non-limiting example, the carotenoid composition can comprise the meso-zeaxanthin diacetate, (3R,3′R,6R)-lutein (free, ester, and/or diacetate form), and (3R,3′R)-zeaxanthin (free, ester, and/or diacetate form) in a meso-zeaxanthin diacetate:(3R,3′R,6R)-lutein:(3R,3′R)-zeaxanthin ratio of about 10:10:2. In other aspects, each xanthophyll carotenoid present in the carotenoid composition is independently and individually included at a concentration of greater than or equal to about 1% (w/w) to less than or equal to about 30% (w/w).

The carotenoid composition is free, or substantially free, of water, where “substantially free of water” means that the water can be included at a concentration that is too low to form an emulsion, such as at a concentration of less than or equal to about 5% (w/w) or less than or equal to about 2.5% (w/w) (based on the total weight of the carotenoid composition). As such, in some aspects, the carotenoid composition includes 0% (w/w), about 0.001% (w/w), about 0.05% (w/w), about 0.5% (w/w), about 1% (w/w), about 1.5% (w/w), about 2% (w/w), about 2.5% (w/w), about 3% (w/w), about 3.5% (w/w), about 4% (w/w), about 4.5% (w/w), or about 5% (w/w) water, which may be unintentionally included as a result of humidity. Accordingly, in some aspects, the carotenoid composition is not an emulsion; rather, it is a homogenous or uniform composition without noticeable, i.e., observable, differing phases. Alternatively, the carotenoid composition can be a suspension of non-micellar particles in a lipid matrix defined at least partially by the phospholipids.

In certain aspects, the carotenoid composition is in the form of a tablet or a capsule (such as a gel capsule) having a mass of greater than or equal to about 250 mg to less than or equal to about 750 mg, such as a mass of about 250 mg, about 300 mg, about 350 mg, about 400 mg, about 450 mg, about 500 mg, about 550 mg, about 600 mg, about 650 mg, about 700 mg, or about 750 mg. The tablet or capsule comprises greater than or equal to about 0.5 mg to less than or equal to about 20 mg of the each xanthophyll carotenoid diacetate present. In a non-limiting example, the tablet or capsule comprises about 10 mg meso-zeaxanthin diacetate, about 10 mg (3R,3′R,6R)-lutein (in base, ester, and/or diacetate form), and about 2 mg (3R,3′R)-zeaxanthin (in base, ester, and/or diacetate form).

The phospholipids are included in the carotenoid composition at a concentration of greater than or equal to about 0.1% (w/w) to less than or equal to about 10% (w/w), greater than or equal to about 0.1% (w/w) to less than or equal to about 5% (w/w), or greater than or equal to about 0.1% (w/w) to less than or equal to about 2.5% (w/w). The phospholipids can include any amphipathic phospholipid molecule known in the art capable of forming a micelle. Non-limiting examples of such molecules include phosphatidylcholines, lysophosphatidylcholines, phosphatidic acids, phosphatidylethanolamines, phosphatidylglycerols, phosphatidylserines, phosphoinositides, phosphosphingolipids, and combinations thereof.

Non-limiting examples of phosphatidylcholines include 1,2-Didecanoyl-sn-glycero-3-phosphocholine (DDPC), 1,2-Dierucoyl-sn-glycero-3-phosphocholine (DEPC), 1,2-Dilinoleoyl-sn-glycero-3-phosphocholine (DLOPC), 1,2-Dilauroyl-sn-glycero-3-phosphocholine (DLPC), 1,2-Dimyristoyl-sn-glycero-3-phosphocholine (DMPC), 1,2-Dioleoyl-sn-glycero-3-phosphocholine (DOPC), 1,2-Dipalmitoyl-sn-glycero-3-phosphocholine (DPPC), 1,2-Distearoyl-sn-glycero-3-phosphocholine (DSPC), Egg-PC(EPC), Hydrogenated Egg PC (HEPC), High purity Hydrogenated Soy PC (HSPC), Hydrogenated Soy PC (HSPC), 1-Myristoyl-2-palmitoyl-sn-glycero 3-phosphocholine (Milk Sphingomyelin MPPC), 1-Myristoyl-2-stearoyl-sn-glycero-3-phosphocholine (MSPC), 1-Palmitoyl-2-myristoyl-sn-glycero-3-phosphocholine (PMPC), 1-Palmitoyl-2-oleoyl-sn-glycero-3-phosphocholine (POPC), 1-Palmitoyl-2-stearoyl-sn-glycero-3-phosphocholine (PSPC), 1-Stearoyl-2-myristoyl-sn-glycero-3-phosphocholine (SMPC), 1-Stearoyl-2-oleoyl-sn-glycero-3-phosphocholine (SOPC), 1-Stearoyl-2-palmitoyl-sn-glycero-3-phosphocholine (SPPC), and combinations thereof.

Non-limiting examples of lysophosphatidylcholines include 1-Myristoyl-sn-glycero-3-phosphocholine (LYSOPC MYRISTIC), 1-Palmitoyl-sn-glycero-3-phosphocholine (LYSOPC PALMITIC), 1-Stearoyl-sn-glycero-3-phosphocholine (LYLSOPC STEARIC), and combinations thereof.

Non-limiting examples of phosphatidic acids include 1,2-Dierucoyl-sn-glycero-3-phosphate (Sodium Salt) (DEPA-NA), 1,2-Dilauroyl-sn-glycero-3-phosphate (Sodium Salt) (DLPA-NA), 1,2-Dimyristoyl-sn-glycero-3-phosphate (Sodium Salt) (DMPA-NA), 1,2-Dioleoyl-sn-glycero-3-phosphate (Sodium Salt) (DOPA-NA), 1,2-Dipalmitoyl-sn-glycero-3-phosphate (Sodium Salt) (DPPA-NA), 1,2-Distearoyl-sn-glycero-3-phosphate (Sodium Salt) (DSPA-NA), and combinations thereof.

Non-limiting examples of phosphatidylethanolamines include 1,2-Dierucoyl-sn-glycero-3-phosphoethanolamine (DEPE), 1,2-Dilauroyl-sn-glycero-3-phosphoethanolamine (DLPE), 1,2-Dimyristoyl-sn-glycero-3-phosphoethanolamine (DMPE), 1,2-Dioleoyl-sn-glycero-3-phosphoethanolamine (DOPE), 1,2-Dipalmitoyl-sn-glycero-3-phosphoethanolamine (DPPE), 1,2-Distearoyl-sn-glycero-3-phosphoethanolamine (DSPE), 1-Palmitoyl-2-oleoyl-sn-glycero-3-phosphoethanolamine (POPE), and combinations thereof.

Non-limiting examples of phosphatidylglycerols include 1,2-Dierucoyl-sn-glycero-3[Phospho-rac-(1-glycerol . . . ) (Sodium Salt) (DEPG-NA), 1,2-Dilauroyl-sn-glycero-3[Phospho-rac-(1-glycerol . . . ) (Sodium Salt) (DLPG-NA), 1,2-Dilauroyl-sn-glycero-3[Phospho-rac-(1-glycerol . . . ) (Ammonium Salt) (DLPG-NH4, 1,2-Dimyristoyl-sn-glycero-3[Phospho-rac-(1-glycerol . . . ) (Sodium Salt) (DMPG-NA), 1,2-Dimyristoyl-sn-glycero-3[Phospho-rac-(1-glycerol . . . ) (Ammonium Salt) (DMPG-NH4), 1,2-Dimyristoyl-sn-glycero-3[Phospho-rac-(1-glycerol . . . ) (Sodium/Ammonium Salt) (DMPG-NH4/NA), 1,2-Dioleoyl-sn-glycero-3[Phospho-rac-(1-glycerol . . . ) (Sodium Salt) (DOPG-NA), 1,2-Dipalmitoyl-sn-glycero-3[Phospho-rac-(1-glycerol . . . ) (Sodium Salt) (DPPG-NA), 1,2-Dipalmitoyl-sn-glycero-3[Phospho-rac-(1-glycerol . . . ) (Ammonium Salt) (DPPG-NH4), 1,2-Distearoyl-sn-glycero-3[Phospho-rac-(1-glycerol . . . ) (Sodium Salt) (DSPG-NA), 1,2-Distearoyl-sn-glycero-3[Phospho-rac-(1-glycerol . . . ) (Ammonium Salt) (DSPG-NH4), 1-Palmitoyl-2-oleoyl-sn-glycero-3[Phospho-rac-(1-glycerol) . . . ] (Sodium Salt) (POPG-NA), and combinations thereof.

Non-limiting examples of phosphatidylserines include 1,2-Dilauroyl-sn-glycero-3-phosphoserine (Sodium Salt) (DLPS-NA), 1,2-Dimyristoyl-sn-glycero-3-phosphoserine (Sodium Salt) (DMPS-NA), 1,2-Dioleoyl-sn-glycero-3-phosphoserine (Sodium Salt) (DOPS-NA), 1,2-Dipalmitoyl-sn-glycero-3-phosphoserine (Sodium Salt) (DPPS-NA), 1,2-Distearoyl-sn-glycero-3-phosphoserine (Sodium Salt) (DSPS-NA), and combinations thereof.

Non-limiting examples of phosphoinositides include phosphatidylinositol (PI), phosphatidylinositol 4-phosphate (PIP4), phosphatidylinositol 5-phosphate (PIP5), phosphatidylinositol 3,4-bisphosphate (PI(3,4)P2), phosphatidylinositol 3,5-bisphosphate (PI(3,5)P2), phosphatidylinositol 4,5-bisphosphate (PI(4,5)P2), phosphatidylinositol 3,4,5-triphosphate (PI(3,4,5)P3), and combinations thereof.

Non-limiting examples of phosphosphingolipids include ceramide phosphorylcholine (sphingomyelin) (SPH), ceramide phosphorylethanolamine (sphingomyelin) (Cer-PE), ceramide phosphoryllipid, cerebrosides, gangliosides, and combinations thereof.

The composition is either free or substantially free of micelles or free or substantially free of micelles encapsulating the xanthophyll carotenoid diacetate or the plurality of xanthophyll carotenoid diacetates when more than one xanthophyll carotenoid diacetate is present. By “substantially free,” it is meant that the composition does not contain intentionally added or intentionally formed micelles, although there may be low levels of micelles present. For example, the composition that is substantially free of micelles may include less than or equal to about 5% (w/w) micelles (based on the total weight of the carotenoid composition).

However, the composition is configured to form micelles encapsulating the xanthophyll carotenoid diacetate when subjected to acidic conditions, such as those found within a digestive tract, and more particularly, those found within a stomach and/or small intestine. These acidic conditions include a pH of less than or equal to about 3.5, less than or equal to about 3, or less than or equal to about 2. The pH can be within a pH range of greater than or equal to about 1 to less than or equal to about 3.5 or greater than or equal to about 2 to less than or equal to about 3. Exemplary acid pHs are about 1, about 1.5, about 2, about 2.5, about 3, and about 3.5. The acidic conditions also include a temperature of greater than or equal to about 30° C. to less than or equal to about 45° C., including temperature of about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., and about 45° C. Accordingly, the composition is configured such that micelles encapsulating the xanthophyll carotenoid diacetate and optionally encapsulating the at least one other xanthophyll carotenoid in base form, ester form, diacetate form, or combinations thereof are formed in a digestive tract of a subject after it is orally administered to the subject, i.e., consumed or swallowed. The micelles formed from the composition in the digestive tract have an average diameter of greater than or equal to about 10 nm to less than or equal to about 500 nm, greater than or equal to about 20 nm to less than or equal to about 250 nm, or greater than or equal to about 40 nm to less than or equal to about 100 nm.

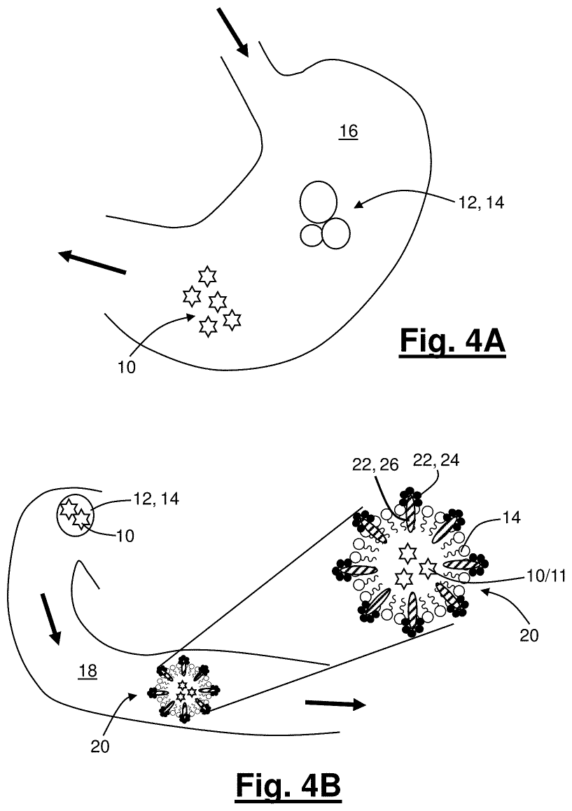

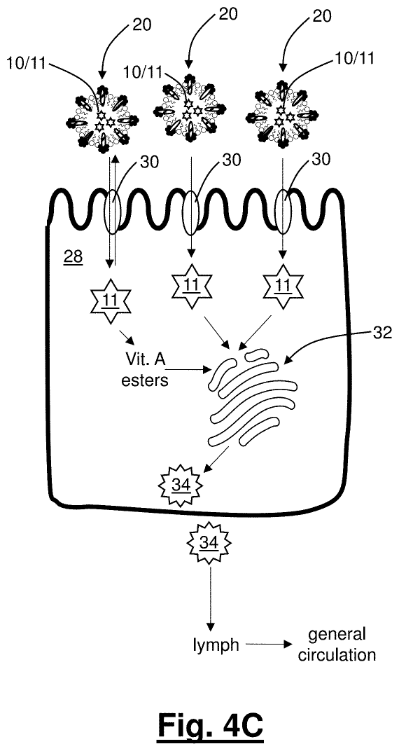

A process of digestion and micelle formation is illustrated in FIG. 4A. It is shown that after oral administration of the xanthophyll carotenoid composition, xanthophyll carotenoid diacetates 10 and lipid droplets 12, comprising phospholipids 14 from the xanthophyll carotenoid composition, are released in the stomach 16. The xanthophyll carotenoid diacetates 10 form large drops of fat with other lipophilic molecules, such as cholesterol and fatty acids. As shown in FIG. 4B, a mixture of water with the xanthophyll carotenoid diacetates 10 in the lipid droplets 12 move to the duodenum 18 of the small intestine, where dissolution (i.e., distribution or spreading out) and absorption via the formation of micelles 20 occur. In the duodenum 18, pancreatic lipases and pancreatic carboxyl ester lipase (CEL) hydrolyze at least a portion of the xanthophyll carotenoid diacetates 10 to their free forms 11, which are more efficiently incorporated into and transported in the micelles 20. However, the xanthophyll carotenoid diacetates 10 and esters may also be encapsulated by the micelles 20. Additionally, bile acids 22, comprising a hydrophilic portion 24 and a hydrophobic portion 26 combine with the phospholipids 14 to form the micelles 20 containing the xanthophyll carotenoid diacetates 10 and free forms 11 when a critical micellar concentration (CMC) of lipophilic molecules (phospholipids 14, bile acids 22, and the like) is reached. Although the phospholipids 14 are shown as having a single hydrophobic tail, it is understood that the phospholipids can also include two tails. Next, cellular uptake, i.e., absorption or assimilation, of the micelles 20 containing the xanthophyll carotenoid diacetates 10 occurs. The cellular uptake is mediated by both passive transport and receptor-mediated (active) transport via SRB1 receptors. As shown in FIG. 4C, the micelles contact an enterocyte 28 and the xanthophyll carotenoid diacetates 10 and/or free forms 11 enter the enterocyte 28 by way of transporter proteins 30. Although the xanthophyll carotenoid free forms 11 are taken up by the enterocyte 28, the xanthophyll carotenoid diacetates 10 and esters also diffuse into the enterocyte 28. The xanthophyll carotenoids, including the xanthophyll carotenoid diacetates 10 and free forms 11, are charged by a Golgi apparatus 32 and released into the lymphatic system as chylomicrons 34.

In certain aspects, a single micelle can encapsulate a plurality of molecules of a single xanthophyll carotenoid or a plurality of molecules of at least two different xanthophyll carotenoids, wherein at least one xanthophyll carotenoid is in the diacetate form. Accordingly, the carotenoid composition provides improved bioavailability relative to a corresponding carotenoid composition that comprises micelles encapsulating the xanthophyll carotenoid diacetate, including compositions comprising micelles encapsulating meso-zeaxanthin diacetate as a xanthophyll carotenoid at a concentration of greater than or equal to about 5% (w/w) (based on the total weight of the carotenoid composition).

In various aspects, the composition further comprises an emulsifying and stabilizing agent, such as at least one surfactant, at a concentration of greater than or equal to about 1% (w/w) to less than or equal to about 10% (w/w). Non-limiting examples of the emulsifying and stabilizing agent include gum Arabic, gum xanthan, guar gum, alginate, pectin, a polysorbate (e.g., polysorbate 80, polysorbate 65, polysorbate 60, polysorbate 20, and combinations thereof, including Tween® polysorbates 80, 65, 60, and/or 20), oleic acid, medium chain triglycerides, monoglycerides, diglycerides, polyglycerol polyricinoleate, sucrose distearate, sorbitan (e.g., sorbitan stearate, sorbitan laurate, sorbitan sesquioleate, sorbitan oleate, sorbitan tristearate, sorbitan palmitate, sorbitan trioleate, and combinations thereof), and combinations thereof.

In various other aspects, the composition optionally further comprises an antioxidant. The antioxidant can be vitamin E, vitamin C, ascorbyl palmitate, rosemary extract, citric acid, ascorbic acid, tartaric acid, butylated hydroxyanisole (BHA), butylated hydroxytoluene (BHT), potassium sorbate, or combinations thereof, as non-limiting examples. Accordingly, the composition can include at least one antioxidant. When present, each antioxidant is individually and independently included at a concentration of greater than or equal to about 20 mg/kg to less than or equal to about 20 g/kg, greater than or equal to about 20 mg/kg to less than or equal to about 10 g/kg, or greater than or equal to about 10 IU to less than or equal to about 500 IU.

In various other aspects, the composition further comprises an alkali metal, an alkaline earth metal, or a transition metal or transition metal ion, such as zinc (Zn), zinc ions (e.g., Zn²⁺), copper (Cu), copper ions (e.g., Cu²⁺, Cu³⁺, or combinations thereof), manganese ions, iron ions, or combinations thereof, as non-limiting examples. The alkali metal can be provided as an ion or salt of lithium (Li, Li⁺), sodium (Na, Na⁺), or potassium (K, K⁺). The alkaline earth metal can be provided as an ion or salt of magnesium (Mg, Mg²⁺) or calcium (Ca, Ca²⁺). The transition metal ions can be provided, for example, as transition metal salts, including oxides, sulfates, chlorides, gluconates, stearates, and combinations thereof, as non-limiting examples. Exemplary transition metal salts include zinc oxide, cupric oxide, cuprous oxide, iron oxide, and combinations thereof. Accordingly, the composition can include at least one transition metal salt. When present, alkali metal salt, alkaline earth metal salt, and/or the transition metal salt is present in the composition at individual and independent concentrations of greater than or equal to about 2 mg/kg to less than or equal to about 200 mg/kg or greater than or equal to about 20 mg/kg to less than or equal to about 100 mg/kg.

The carotenoid composition also includes a carrier at a concentration of greater than or equal to about 50% (w/w) to less than or equal to about 90% (w/w) (based on the total weight of the carotenoid composition). As non-limiting examples, the carrier can include a plant extract (such as sunflower oil, soybean oil, canola oil, corn oil, safflower oil, olive oil, citrus oil, or combinations thereof, as non-limiting examples), a vegetable oil, a mineral oil, an animal oil (such as fish oil), or combinations thereof. Other carriers include glycerine, gelatin (for example, beef and/or pork gelatin), beeswax, and fatty acids. In certain aspects, the carotenoid composition is substantially free of gluten.

Methods of making the diacetates are described in U.S. Pat. No. 5,959,138, which is incorporated herein by reference in its entirety.

The current technology also provides a method of supporting good eye health in a subject in need thereof. As used herein, supporting good eye health includes supplementing carotenoids in the macula, restoring lowered (e.g., below normal levels) carotenoid levels in the subject, and/or restoring macular pigment (such as below-normal levels of macular pigment) in the subject. As such, the method treats AMD, slows the progression of AMD, or minimizes the chances of acquiring (or preventing) AMD or other macula-related conditions. The subject can be a human or non-human mammal having AMD or at risk of having AMD. In certain aspects, the composition maintains, supports, or enhances vision in the subject.

The method comprises administering to the subject a safe and therapeutically effective amount of the carotenoid composition described herein. As used herein, the term “therapeutically effective amount” means an amount of a compound that when administered to a subject having AMD, at risk of having AMD, or desiring to support macular health is sufficient, either alone or in combination with additional therapies, to effect treatment of the AMD or to otherwise provide the macula with supporting levels of at least one carotenoid. The “therapeutically effective amount” will vary depending on, for example, the compound, pharmaceutical composition or pharmaceutical dosage form, the condition treated and its severity, and the age and weight of the patient to be treated. In various aspects, a therapeutically effective amount of the composition provides a dose of each included carotenoid of greater than or equal to about 0.5 mg to less than or equal to about 25 mg.

The current technology also provides a method of improving the bioavailability of a xanthophyll carotenoid, such as meso-zeaxanthin, as a non-limiting example, in a subject. The subject can be a human or non-human mammalian subject desiring to support good eye health, having AMD, at risk of having AMD, or having or at risk of having another macula-related condition.

The method comprises converting a xanthophyll carotenoid diacetate into xanthophyll carotenoid in free form in the digestive tract of the subject and forming micelles within the digestive tract (such as in an acid environment provided by the stomach and/or small intestine) of the subject, the micelles comprising a monolayer of phospholipids encapsulating the xanthophyll carotenoid in free form. By forming the micelles comprising the xanthophyll carotenoid in the digestive track, more of the xanthophyll carotenoid is biologically available within the blood stream of the subject than would be biologically available if the xanthophyll carotenoid was not packaged into a micelle in the digestive tract of the subject. As such, more of the administered xanthophyll carotenoid remains available to the macula of the subject than would be available if administered in crystalline form.

The formation of micelles within the digestive tract of the subject is a result of administering a safe and effective amount of a carotenoid composition to the subject, as discussed above. Accordingly, the carotenoid composition can be any carotenoid composition described herein.

Embodiments of the present technology are further illustrated through the following non-limiting examples.

### Example 1

(3R,3′R,6R)-lutein (L), (3R,3′R)-zeaxanthin (Z), and meso-zeaxanthin (MZ) have been the focus of research and commercial interest for their applications in human health. Research into formulations to enhance their bioavailability is merited. This 6-month randomized placebo-controlled trial involving 81 healthy volunteers compared the bioavailability of five different formulations of L, Z, and MZ crystals in sunflower or omega-3 oil versus L, Z, and MZ diacetates (Ld, Zd, and MZd) in a micromicellar-precursor formulation, wherein the term “micromicellar-precursor” reflects the ability of the formulation to form micelles containing xanthophyll carotenoids in free form within a subject's digestive tract. Fasting serum carotenoids, macular pigment, and skin carotenoid scores were analyzed at baseline and 6 months. Serum L, Z, and MZ concentrations increased in all active interventions compared to placebo (p<0.001 to p=0.008). The diacetate micromicelle-precursor formulation exhibited a significantly higher mean response in serum concentrations of Z and MZ compared to the other active interventions (p=0.002-0.019). A micromicellar-precursor formulation with solubilized Z and MZ diacetates is a technology advance that enhances the bioavailability of these carotenoids when compared to traditional carotenoid formulations.

### Introduction

L, Z, and MZ are xanthophyll carotenoids (XC) that singularly deposit in the human macula lutea, where they are known as macular pigment (MP). L and Z are obtained solely through dietary intake. MZ may be obtained from endogenous conversion of L in the retinal pigment epithelium, but it can also be found in trace amounts in diet. Over the last two decades, intervention trials have studied the role of L, Z, and MZ in human health using nutritional supplements. Reports confirm that these carotenoids enhance visual performance and cognitive function and are potential preventive and therapeutic agents in retinal pathology, such as non-advanced AMD.

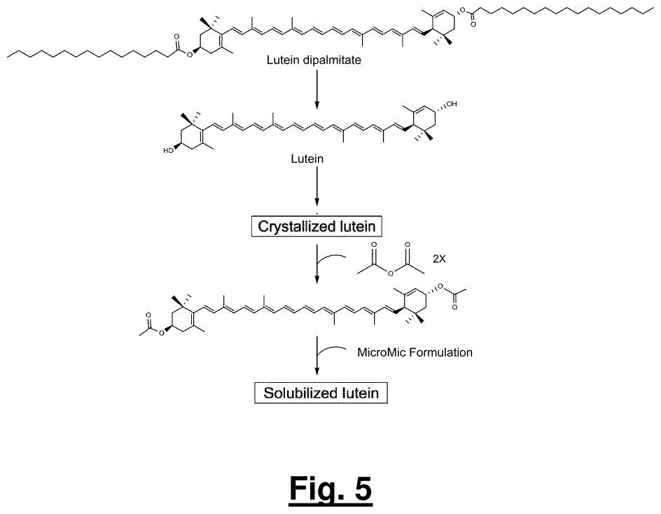

L used in nutritional supplements is extracted from the marigold flower (Tagetes erecta L.), while Z is obtained from specific varieties of this flower and peppers. MZ is obtained from L through a process that promotes the migration of a double bond that turns the c-ring of L into a p-ring. In every case, the final purification step forms XC microcrystals, which are further processed to generate solubilized XCs. (FIG. 5). Nutraceutical companies continually seek to develop new methods to protect these microcrystals from oxidation, improve their solubility in aqueous matrices, and increase their bioavailability in the digestive system. Among the most common methods to protect the XC microcrystals is dispersion in edible oils or encapsulation with biopolymers. To increase bioavailability and solubility in different matrices, researchers emulsify the XC following different methods. However, none of these methods managed to dissolve the microcrystals completely. Recently, a new method esterifying the XC with short organic acids claimed to keep XC solubilized without the formation of microcrystals under environmental conditions of temperature and pressure. In this process, XC are esterified with acetate or propionate to form L, Z, and MZ diacetates (Ld, Zd, and MZd, respectively). After this reaction takes place, XC derivatives are then homogenized in their natural original flower matrix in the presence of lipids, phospholipids, fatty acids, and emulsifiers to keep XC soluble. In the digestive system, this soluble state facilitates the incorporation of XC into micromicelles, which are spherical aggregates of lipid molecules in the presence of amphiphilic compounds known as surfactants. This formulation has been previously tested in clinical trials and compared to crystallized formulations (free L).

This example presents findings of the Carotenoid-Omega Availability Study (COAST), which was performed to compare the bioavailability of Ld, Zd and MZd in a micromicelle-precursor formulation with classical formulations containing free carotenoids as microcrystals suspended in oil.

### Materials and Methods

Design and Study Population.

COAST was a 6-month, double-blind, block-randomized placebo-controlled study involving 81 healthy participants between 18 and 65 years old. Participant recruitment and assessment commenced on December 2017 and ended on December 2018. Recruitment was achieved through local media and advertisement at the Waterford Institute of Technology, local fitness centers, and with employees of an industry based in Waterford, Ireland. Participants were excluded if they had a medical diagnosis of a critical or acute medical condition and/or if they were taking nutritional supplements containing L, Z, MZ, or omega-3 fatty acids. Every participant enrolled in the study provided written informed consent prior to commencement. The study protocol was approved May 2017 by the Research Ethics Committees of the Waterford Institute of Technology (Waterford, Ireland) and the HSE, South Eastern Area (University Hospital Waterford, Waterford, Ireland). Industrial Orgánica, S.A. de C.V., the manufacturer of the nutritional supplements, had no role in the design of the study, the collection and analysis of the data, or the preparation of manuscripts. All vouch for the accuracy of the data and the fidelity of the study to the protocol.

Interventions.

COAST was a five-arm intervention study, where participants were randomly allocated, with equal probability and separately for men and women, to one of four active intervention groups or to a placebo group. Label claims of the nutritional content in the intervention supplements were as follows: Group 1, L (10 mg)+MZ (10 mg)+Z (2 mg) provided in one capsule; Group 2, L (10 mg)+MZ (10 mg)+Z (2 mg) provided in two capsules; Group 3, L (10 mg)+MZ (10 mg)+Z (2 mg) provided in DHA (430 mg) and EPA (90 mg) in two capsules; and Group 4, Ld (10 mg)+MZd (10 mg)+Zd (2 mg) provided in a micromicelle-precursor formulation in one capsule or Group 5, placebo (sunflower oil). Of note, analysis of the supplements per group conducted at the laboratory showed slightly different carotenoid concentrations to label claim (see Table 1). A statistical analysis conducted to compare results of the analyzed concentrations to those in label claim did not show significantly different results. Therefore, it was decided to present the dosages of the formulations as stated by label claim. L, Z, and MZ were supplied in free form in a sunflower oil suspension for all except for Group 4, which was supplied as L, Z, and MZ diacetates in a solubilizate prepared for micellarization. L, Z, and MZ in Group 3 were dissolved in DHA and EPA supplied by Epax (Alesund, Norway; product number: EPAX1050TG). Vitamin E (DL-α-tocopheryl acetate; 5 g/kg) was added as a preservative. The supplements were provided to the participants in a sealed container and the capsules for all the intervention groups were identical in appearance. Subjects were instructed to take either 1 or 2 capsules per day depending on the intervention with a meal. The supplements were provided by Industrial Organica (Monterrey, Mexico) free-of-charge for use in the trial.

TABLE 1
Carotenoid concentrations analyzed per capsule interventions¹.

| | | | | | Group |
|---|---|---|---|---|---|
| Carotenoid | Group 1 | Group 2 | Group 3 | Group 4 | 5 |
| Lutein | 9.42 ± 0.11 | 5.80 ± 0.19 | 4.48 ± 0.07 | 10.24 ± 0.54 | 0 |
| Meso- | 13.06 ± 0.15 | 8.12 ± 0.27 | 6.49 ± 0.12 | 10.62 ± 0.61 | 0 |
| zeaxanthin | | | | | |
| Zeaxanthin | 2.12 ± 0.03 | 1.38 ± 0.04 | 1.75 ± 0.03 | 1.98 ± 0.11 | 0 |
| Dosage | 1 | 2 | 2 | 1 | 1 |
| (capsule/day) | | | | | |
| Total | 24.60 | 30.60 | 25.44 | 22.84 | 0 |
| carotenoids | | | | | |
| ingested per | | | | | |
| day (mg) | | | | | |

¹Plus-minus values are means ± SD. Values are total carotenoid concentrations per capsule (mg). There were no significant between-group differences in change of L serum concentrations per gram taken (p = 0.419); change in Z, and MZ serum concentrations per gram taken were higher for Group 4 (p < 0.001). P values were based on chi square and ANOVA or Kruskall-Wallis where appropriate. Bonferroni correction was performed for post-hoc analysis.
Label claim for total nutrient concentrations were as follows: Group 1, L (10 mg) + MZ (10 mg) + Z (2 mg) provided in one capsule; Group 2, L (10 mg) + MZ (10 mg) + Z (2 mg) provided in two capsules; Group 3, L (10 mg) + MZ (10 mg) + Z (2 mg) provided in DHA (430 mg) and EPA (90 mg) in two capsules; and Group 4, Ld (10 mg) + MZd (10 mg) + Zd (2 mg) provided in one capsule; or Group 5, placebo (sunflower oil).

Study Evaluations.

Demographic, lifestyle, medical, and dietary assessment. Standardized case report forms were used to record demographics, lifestyle, medical history, and anthropometrics at two time points, at baseline and at 6 months following supplementation. Cigarette smoking was recorded by smoking status as follows: never, if never smoked more than 100 cigarettes; former, if smoked more than 100 cigarettes in the past year and none in the last month; or current. Education was recorded as high school or less, bachelor's degree, or postgraduate education. Physical examination included height and body weight to calculate body mass index (BMI, kg/m²). International cut-offs for normal, overweight, and obesity were used.

Outcome measures. The primary outcome of the study was the measurement of L, Z, and MZ bioavailability as a response in serum and tissue concentrations. Serum carotenoid concentrations were analyzed as the total concentrations (μmol/L) for each of the carotenoids in serum. Total carotenoid concentrations were obtained by the sum of L, Z, and MZ concentrations. Tissue concentrations of L, Z, and MZ were measured as a composite MP and skin carotenoid score. All methods are described below. Outcome variables were recorded at baseline and at 6 months.

MP measurement. MP was measured by dual-wavelength AF using the Spectralis investigational MP optical density (MPOD) module (Heidelberg Engineering GmbH, Heidelberg, Germany). Specifications and details on the technique and image acquisition have been described. In short, pupils were dilated prior to MP measurement, and patient details were entered into the Heidelberg Eye Explorer (HEYEX version 1.7.1.0) software. Alignment, focus and camera sensitivity were first optimized in near-infrared reflectance mode. Subsequently, BAF+GAF (simultaneous blue and green AF) movie images were acquired, while the HEYEX software ensured proper alignment and averaging of these images in order to generate a MP density map, where the reference eccentricity was defined at 7° retinal eccentricity from point of fixation (where MPOD was defined as zero). MP measurement is reported in terms of MPOV, as standardized previously.

Skin carotenoid concentrations. Total carotenoid concentrations in the skin were obtained using the Nu Skin Pharmanex S3 scanner, a non-invasive instrument that uses Raman spectroscopy technology. This technique generates a skin carotenoid score (SCS) by measuring skin carotenoid concentrations between the maximal and distal palmar creases, directly below the fifth finger of the right hand using the Pharmanex BioPhotonic Scanner device.

Carotenoid serum concentrations. Fasting (overnight fast for greater than 9 hours) blood samples were collected at 0, 3, and 6 months for XC serum analysis. Blood samples were collected by standard venipuncture techniques in 9 mL blood collection tubes (BD Vacutainer SST Serum Separation Tubes) containing a “Z Serum Sep Clot Activator.” Collection tubes underwent thorough mixing of the clot activator. The blood samples were left for 30 minutes at room temperature to clot and then centrifuged at 725 g for 10 minutes in a GruppeGC12 centrifuge (Desaga Sarstedt) to separate the serum from the whole blood. Following centrifugation, serum was transferred to light-resistant microtubes and stored at circa −80° C. until the time of batch analysis. Serum carotenoid analysis was performed by high performance liquid chromatography (HPLC), using a method previously described. Calibration lines used, as well as lower and upper limits of quantification (LLOQ and ULOQ respectively), are as previously performed. Serum carotenoid analysis was completed in sixteen independent batches, with a maximum intra-day precision of 7.28%, measured as RSD, and an inter-day precision of 3.16% (RSD).

Carotenoid content of the supplements used in this study was analyzed following known protocols. For carotenoid content of the formulation containing diacetate-carotenoids, the mobile phase has to be adjusted to a hexane:isopropanol ratio of 99:1 (v/v).

Follow-Up and Adherence.

Follow-up study visits were scheduled at 3 months after baseline and at 6 months (endpoint). Adherence to the treatment regime was assessed by pill count each visit and by serum analysis at 3 months. Information on change in lifestyle and health, as well as adverse events, was collected at each visit. Adverse events were collected through a non-validated questionnaire.

Statistical Analysis.

Data was described using usual statistics, including means (±SDs), medians, minimum, and maximum values for quantitative variables, and frequencies and percentages for qualitative variables. Between-group differences at baseline were analyzed using analysis of variance, or Kruskal-Wallis, as appropriate for quantitative variables and Chi-square test for qualitative variables. Groups differed significantly with respect to BMI at baseline, which was further controlled using ANCOVA. However, BMI in these models was not significantly related to any outcome variable, so it was subsequently removed from each model. Therefore, the results reported below are all for simpler models, relating change in outcome variables to intervention alone.

General linear models were used to analyze change in primary outcome variables (change in carotenoid serum concentrations, MPOV, and skin carotenoid score). Change was analyzed as the difference of the outcome variable from baseline and 6 months. The hypotheses were (a) that the active intervention groups (unrelated treatments) would all have a higher average response after six months in serum and in tissue concentrations compared with the placebo group and (b) that the diacetate micromicellar-precursor formulation would have a higher average response as compared with the other active interventions. The first of these hypotheses was investigated directly from the fitted linear models, and the second using pairwise comparisons based on 2-tailed independent samples T-tests. No adjustment for multiple comparisons was deemed appropriate. Pearson's coefficient was used to investigate relationships between change in serum and change in tissue of carotenoid concentrations. The statistical package IBM SPSS version 25 (Armonk, N.Y.) was used, and a 5% significance level was applied throughout.

### Results

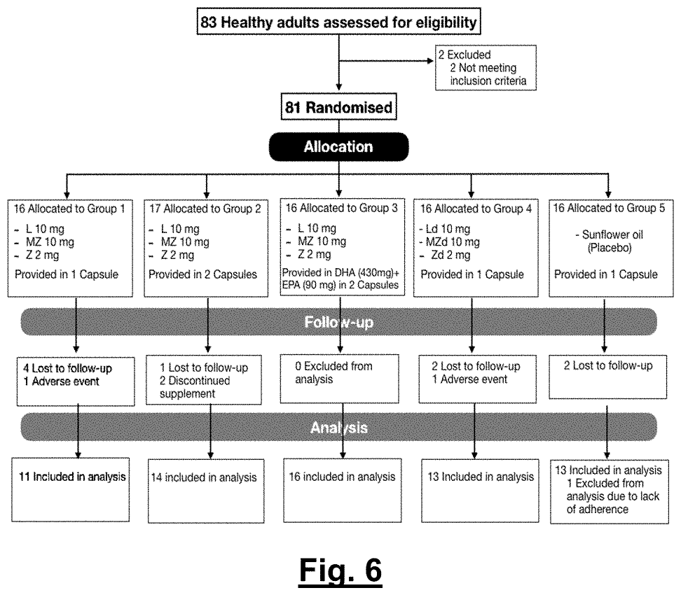

A total of 81 participants were enrolled at baseline with 68 (84%) participants completing final assessment at 6 months; nine (11%) participants were lost to follow-up and four (5%) participants discontinued the study, one due to pregnancy, two due to minor adverse events, and one due to a general practitioner request (see FIG. 6). Adverse events reported throughout the 6 months of the study were all related to minor gastrointestinal symptoms, such as bloating, acid reflux, and discomfort. There was no statistical difference between active interventions and placebo (p>0.05). One participant allocated to the placebo arm was excluded from analysis as they reported supplementation with carotenoids during the duration of the study, which was confirmed by detection of high concentrations of MZ in serum at 6 months.

Baseline Data.

The mean (range) age of the participants was 44.2 (25-62) years, and 50% (n=40) were female. Baseline characteristics were statistically comparable across the five groups, except for BMI (p=0.008), which was within the normal range in Group 2 and Group 5, but higher for Group 1, Group 3, and Group 4 (see Table 2).

The baseline serum and tissue levels of study nutrients were balanced across the treatment groups, as shown in Table 2. MZ concentrations in all participants were undetectable at baseline, which confirms the exclusion criterion of MZ supplementation.

TABLE 2
Baseline characteristics of the study participants¹.

| | | Subjects divided by Intervention Group | | | | |
|---|---|---|---|---|---|---|
| | All Subjects | Group 1 | Group 2 | Group 3 | Group 4 | Group 5 |
| Variable | (n = 80) | (n = 16) | (n = 17) | (n = 16) | (n = 16) | (n = 15) |
| Age (y) | 44.2 ± 10 | 43.8 ± 9.6 | 44.6 ± 9.0 | 41.6 ± 10.7 | 46.7 ± 11.2 | 44.3 ± 9.9 |
| Females, No. (%) | 41 (50.6) | 8 (50) | 9 (53) | 8 (50) | 7 (44) | 8 (50) |
| Smoking, No. (%) | | | | | | |
| Never | 40 (49.4) | 8 (50) | 9 (53) | 8 (50) | 7 (43.7) | 8 (50) |
| Former | 28 (34.6) | 6 (75) | 4 (23.5) | 6 (37.5) | 7 (43.7) | 5 (31.3) |
| Current | 13 (16.0) | 2 (25) | 4 (23.5) | 2 (12.5) | 2 (12.6) | 3 (18.7) |
| Education, No. (%) | | | | | | |
| High-school | 34 (41.9) | 2 (12.5) | 6 (35.3) | 7 (43.7) | 12 (75) | 7 (43.7) |
| College | 31 (38.3) | 10 (62.5) | 6 (35.3) | 6 (37.5) | 3 (18.8) | 6 (37.5) |
| Postgraduate | 16 (19.8) | 4 (25) | 5 (29.4) | 3 (18.8) | 1 (6.2) | 3 (18.8) |
| BMI | 27.3 ± 5.7 | 28.4 ± 6.1 | 24.5 ± 4.5 | 28.7 ± 6.9 | 30.2 ± 5.3 | 25.0 ± 3.3 |
| [range] | [19-43] | [20-42] | [20-38] | [19-43] | [20-39] | [21-30] |

Lutein, zeaxanthin, and meso-zeaxanthin concentrations

| | | | | | | |
|---|---|---|---|---|---|---|
| Serum L, μmol/L | 0.19 ± 0.09 | 0.19 ± 0.06 | 0.21 ± 0.11 | 0.19 ± 0.12 | 0.19 ± 0.10 | 0.18 ± 0.06 |
| Serum Z, μmol/L | 0.076 ± 0.029 | 0.074 ± 0.028 | 0.082 ± 0.037 | 0.071 ± 0.029 | 0.065 ± 0.026 | 0.071 ± 0.021 |
| MPOV | 4575 ± 2222 | 5263 ± 1789 | 4793 ± 2885 | 3890 ± 1925 | 4277 ± 2115 | 4784 ± 1446 |
| [range] | [527-10033] | [2243-8861] | [527-10033] | [1327-7649] | [1027-8639] | [2632-7880] |
| Skin Carotenoid | 35 819 | 37 970 | 40 833 | 34 656 | 30 385 | 36 538 |
| Score | ±11 977 | ±13 652 | ±15 063 | ±11 142 | ±12 065 | ±9 173 |

¹Plus-minus values are means ± SD. There were no significant between-group differences at baseline except for BMI (p = 0.014). P values were based on chi square and ANOVA or Kruskall-Wallis where appropriate.
Abbreviations: L, lutein; Z, zeaxanthin; BMI, body mass index; y, years; MPOV, macular pigment optical volume.
Interventions are as follows: Group 1, L (10 mg) + MZ (10 mg) + Z (2 mg) provided in one capsule; Group 2, L (10 mg) + MZ (10 mg) + Z (2 mg) provided in two capsules; Group 3, L (10 mg) + MZ (10 mg) + Z (2 mg) provided in DHA (430 mg) and EPA (90 mg) in two capsules; and Group 4, Ld (10 mg) + MZd (10 mg) + Zd (2 mg) provided in one capsule; or Group 5, placebo (sunflower oil).

L, Z, and MZ Serum Concentrations.

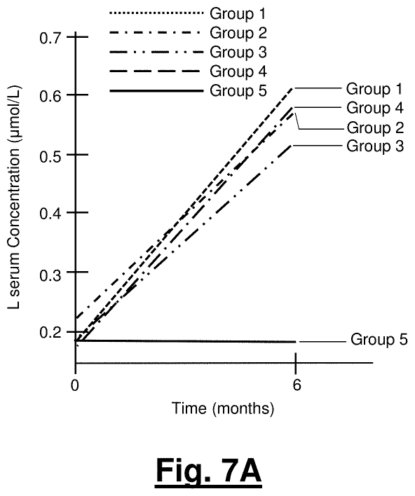

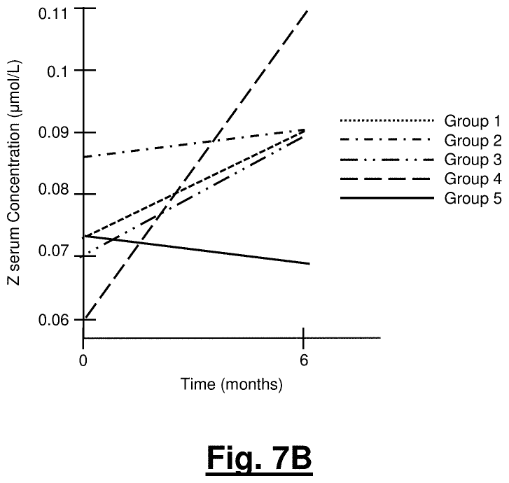

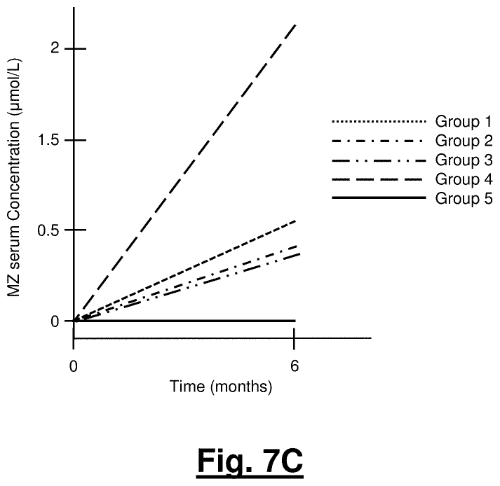

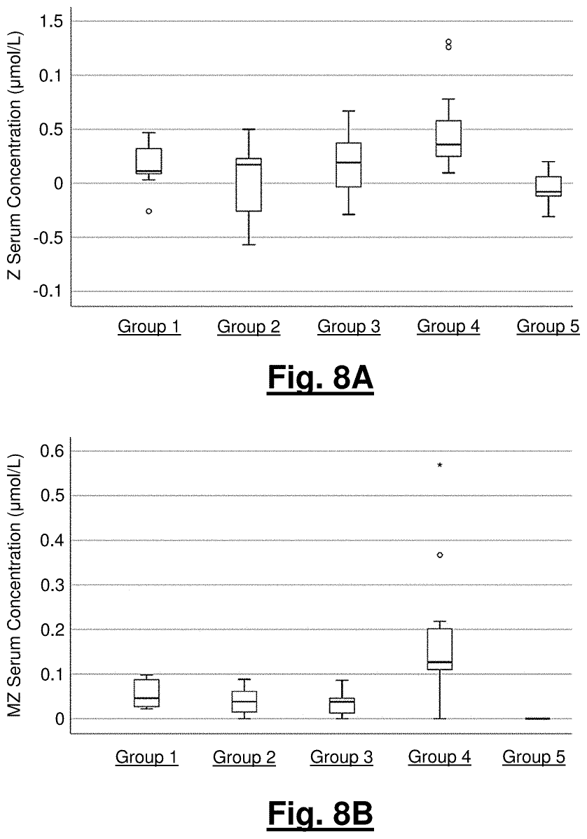

The increase in serum concentrations of L, Z, and MZ in all active groups was statistically significant compared to placebo (p<0.001 to p=0.008) (Table 3), except for Group 2 (p=0.366). Graphs showing the serum concentration of L, Z, and MZ at 0 and 6 months are shown in FIGS. 7A, 7B, and 7C, respectively. In addition, the increase in Z and MZ serum concentrations in Group 4 (diacetate micromicelle-precursor formulation) was significantly greater compared to the other three active groups (p=0.002-0.019). Data is provided in Table 4, and bar graphs comparing the Z and MZ serum concentrations in the groups are shown in FIGS. 8A and 8B, respectively.

TABLE 3
Response in serum and tissue concentrations to different formulations of
nutritional supplements with L, Z, and MZ compared to placebo¹.

| | Outcome (μmol/L) | | | | | |
|---|---|---|---|---|---|---|
| Intervention | | L | Z | MZ | MPOV | Skin |
| Group 1 | 0 Mo | 0.189 ± 0.062 | 0.074 ± 0.028 | 0 | 5263 ± 1789 | 37970 ± 13652 |
| (n = 16) | 6 Mo | 0.609 ± 0.253 | 0.090 ± 0.033 | 0.055 ± 0.031 | 5943 ± 1567 | 52303 ± 15253 |
| | Change | 0.425 ± 0.224 | 0.017 ± 0.02 | 0.055 ± 0.031 | 680 ± 661 | 14333 ± 8467 |
| | p value² | <0.001 | 0.007 | <0.001 | 0.039 | 0.024 |
| Group 2 | 0 Mo | 0.210 ± 0.112 | 0.086 ± 0.038 | 0 | 4793 ± 2885 | 40833 ± 15063 |
| (n = 17) | 6 Mo | 0.565 ± 0.289 | 0.091 ± n0.024 | 0.040 ± 0.03 | 5802 ± 3254 | 48571 ± 10921 |
| | Change | 0.342 ± 0.289 | 0.004 ± 0.032 | 0.040 ± 0.03 | 1010 ± 914 | 7738 ± 9369 |
| | p value² | 0.001 | 0.366 | <0.001 | 0.006 | 0.543 |
| Group 3 | 0 Mo | 0.189 ± 0.116 | 0.071 ± 0.03 | 0 | 3890 ± 1925 | 34656 ± 11142 |
| (n = 16) | 6 Mo | 0.516 ± 0.291 | 0.089 ± 0.031 | 0.037 ± 0.029 | 4911 ± 1846 | 45542 ± 10750 |
| | Change | 0.328 ± 0.25 | 0.019 ± 0.026 | 0.037 ± 0.029 | 1021 ± 743 | 10885 ± 7115 |
| | p value² | <0.001 | 0.008 | <0.001 | 0.001 | 0.087 |
| Group 4 | 0 Mo | 0.185 ± 0.104 | 0.060 ± 0.022 | 0 | 4277 ± 2115 | 30385 ± 12065 |
| (n = 16) | 6 Mo | 0.575 ± 0.435 | 0.109 ± 0.052 | 0.164 ± 0.15 | 5331 ± 2061 | 47718 ± 12718 |
| | Change | 0.398 ± 0.384 | 0.049 ± 0.038 | 0.164 ± 0.15 | 1054 ± 680 | 17333 ± 12664 |
| | p value² | 0.002 | <0.001 | 0.001 | 0.001 | 0.012 |
| Group 5 | 0 Mo | 0.189 ± 0.055 | 0.073 ± 0.021 | 0 | 4784 ± 1446 | 36538 ± 9173 |
| (n = 15) | 6 Mo | 0.182 ± 0.056 | 0.069 ± 0.019 | 0 | 4894 ± 1581 | 14333 ± 8467 |
| | Change | −0.005 ± 0.045 | −0.004 ± 0.014 | 0 | 110 ± 606 | 5538 ± 9125 |
| | p value² | — | — | — | — | — |

¹Values are mean ± SD
L, indicates (3R,3′R,6R)-lutein serum concentrations, μmol/L); Z, indicates zeaxanthin serum concentrations, μmol/L); MZ, indicates meso-zeaxanthin serum concentrations, μmol/L); MPOV, macular pigment optical volume; Skin, indicates skin carotenoid score.
²Between group differences comparing change from baseline in each intervention group with placebo were analyzed with the use of an independent-sample t-test. Change was calculated as the difference from baseline. Interventions are as follows: Group 1, L (10 mg) + MZ (10 mg) + Z (2 mg) provided in one capsule; Group 2, L (10 mg) + MZ (10 mg) + Z (2 mg) provided in two capsules; Group 3, L (10 mg) + MZ (10 mg) + Z (2 mg) provided in DHA (430 mg) and EPA (90 mg) in two capsules; and Group 4, Ld (10 mg) + MZd (10 mg) + Zd (2 mg) provided in one capsule; or Group 5, placebo (sunflower oil).

TABLE 4
Effect of diacetate formulation on serum and tissue compared with the
other interventions¹.

| | Group 4 vs. Group 1 | | Group 4 vs. Group 2 | | Group 4 vs. Group 3 | |
|---|---|---|---|---|---|---|
| Outcome | Difference in change | p-value | Difference in change | p-value | Difference in change | p-value |
| L | −0.027 (−0.297 to 0.244) | 0.839 | 0.056 (−0.208 to 0.320) | 0.666 | 0.070 (−0.175 to 0.315) | 0.563 |
| Z | 0.033 (0.006 to 0.059) | 0.018 | 0.045 (0.018 to 0.072) | 0.002 | 0.030 (0.005 to 0.055) | 0.019 |
| MZ | 0.109 (0.020 to 0.197) | 0.019 | 0.124 (0.036 to 0.211) | 0.009 | 0.126 (0.039 to 0.214) | 0.008 |
| MPOV | 374 (−196 to 944) | 0.187 | 45 (−598 to 687) | 0.888 | 33 (−515 to 581) | 0.903 |
| SKIN | 3000 (−6310 to 12310) | 0.511 | 9595 (811 to 18379) | 0.034 | 6448 (−1191 to 14087) | 0.095 |

¹Values are mean (95% Cl).
Abbreviations: L, lutein (indicates L serum concentrations, μmol/L); Z, zeaxanthin (indicates Z serum concentrations, μmol/L); MZ, meso-zeaxanthin (indicates MZ serum concentrations, μmol/L); MPOV, macular pigment optical volume; Skin, indicates skin carotenoid score.
The between-group differences were analyzed with the use of an independent-sample t-test to compare group 4 against the other 3 active groups. Interventions are as follows: Group 1, L (10 mg) + MZ (10 mg) + Z (2 mg) provided in one capsule; Group 2, L (10 mg) + MZ (10 mg) + Z (2 mg) provided in two capsules; Group 3, L (10 mg) + MZ (10 mg) + Z (2 mg) provided in DHA (430 mg) and EPA (90 mg) in two capsules; and Group 4, Ld (10 mg) + MZd (10 mg) + Zd (2 mg) provided in one capsule; or Group 5, placebo (sunflower oil).

L, Z, and MZ Tissue Concentrations.

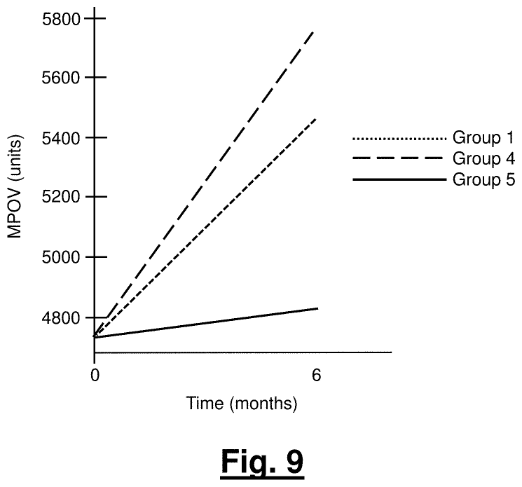

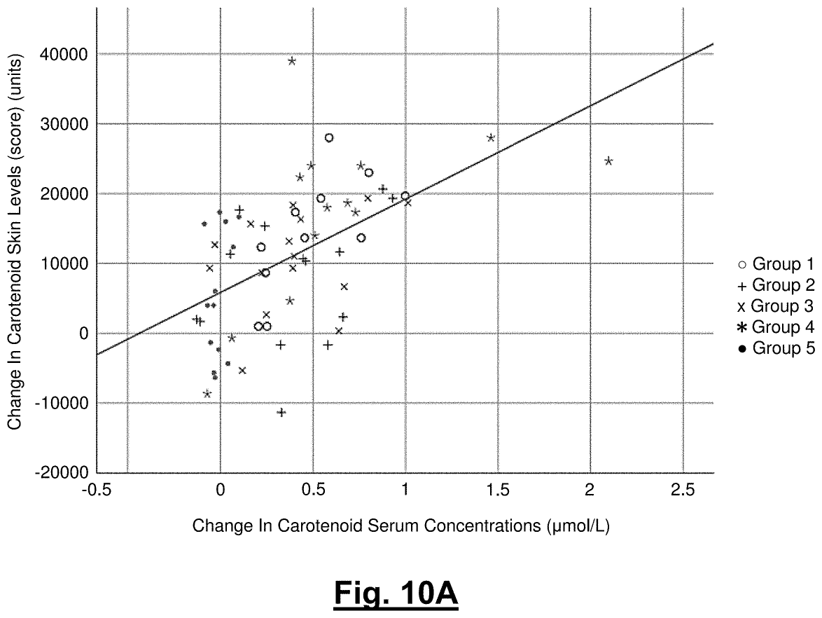

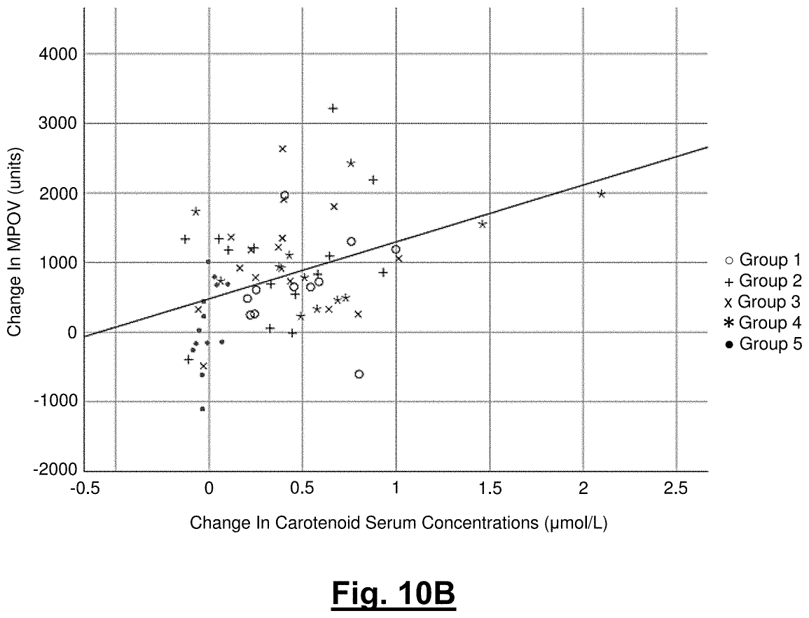

MP and MPOV. The increase in MPOV in all groups was statistically significant compared to placebo (see Table 3). A graph showing the MPOV change over time is shown in FIG. 9. Interestingly, the correlation between change in total serum carotenoids and MPOV was r=0.408, p=0.001 (see FIGS. 10A-10B). There were no significant differences between the four active intervention groups when comparing MPOV improvements.

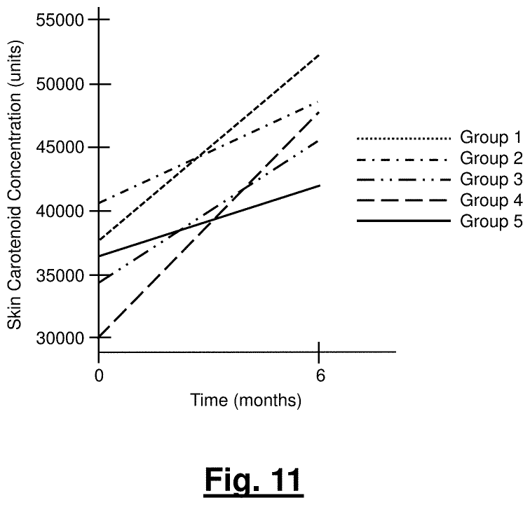

Carotenoid skin concentrations. The change in skin carotenoid concentrations was positively correlated to the total change in serum concentrations (r=0.528, p<0.001) (FIG. 4). The change in skin carotenoid concentrations was statistically significant only in Group 1 and Group 4 compared to placebo (p=0.024 and p=0.012, respectively) (see Table 3). In addition, skin carotenoid score increases in Group 4 were significantly higher compared to Group 2 (p=0.034) (see Table 4). A graph showing the skin carotenoid concentration change over time in Groups 1-5 is shown in FIG. 11.

### Discussion

In this multiple-arm, randomized clinical trial, daily supplementation with L, Z, and MZ combinations using different formulations significantly increased serum concentrations of these nutrients compared to placebo. After 6 months of supplementation, the median serum concentrations of L and Z increased by 202% and 36%, respectively. This dose-response effect is consistent with the ratio of L and Z provided in the supplement (i.e., about 5:1). Also, MZ serum concentrations significantly increased from baseline. These percentage increases in serum were comparable to previous studies using similar carotenoid formulations and amounts. For example, L increased by 200% in the AREDS 2 study and 304% in the CREST AMD study.

The impact of the diacetate micromicelle-precursor formulation on the absorption of the ingested carotenoids is notable. In the present study, the serum response to Zd and MZd (Group 4, pre-solubilized acetate-esterified XC) was significantly greater compared to the formulations containing free carotenoids as crystals. However, it was striking to see that the serum response to Ld remained similar to that of free L. This is consistent with a previous clinical trial, which reported that serum response to Ld was slightly higher and not statistically different when compared to the group supplemented with free L.

After 6 months of supplementation, mean MPOV in tissue significantly increased by 33% on average for all interventions compared to the placebo, but improvement over time between the active interventions was not significantly different (given that MPOV improved in all interventions) (see Table 3). However, it should be noted that the largest increase in MPOV was seen in Groups 2, 3 and 4, which exhibited almost double the MPOV increases of Group 1. Nevertheless, Group 4, which had the lowest amount of carotenoids in the formulation (see Table 1) exhibited the largest response (see Table 3). A longer duration of supplementation is required to assess the long-term differences between these interventions in terms of MPOV response and functional benefits. With respect to skin carotenoid score, statistically significant improvements compared to placebo were seen in Groups 2 and 4 only; however, Group 4 was significantly superior to Group 2 (see Table 4). This finding is likely attributable to the enhanced bioavailability of Zd and MZd achieved in the micromicelle-precursor formulation.

As mentioned above, these results agree with previous reports showing a greater, but non-significant increase of L in serum in subjects supplementing with Ld compared to the free L. A superior and significant response of Ld when compared to free L supplementation in terms of MPOD improvements has been reported. It has been suggested that the older subjects in the study drove this significant increase. Of note, the mean age of the subjects of the present study was 44 years old. Interestingly, a study in hens to study the bioavailability of diacetate carotenoids generated similar results as the present study. In brief, Zd and MZd exhibited a greater capability to increase the deposition of Z and MZ in egg yolk when compared to supplementation with the free form of these carotenoids. As provided above, the increase achieved by Ld was similar to the increase observed with free L.

In this study, the increase over time of the XC concentrations significantly correlated in serum and tissue for all the groups (r=0.408, p=0.001 and r=0.528, p<0.001, respectively). Of note, this is an important result because in previous interventional trials, the change in serum carotenoids poorly correlated with change in MP, something that is also seen in blood/retinal non-responders. Therefore, the current finding supports the intuitive idea that a greater presence of carotenoids in the blood implies a higher occurrence of these nutrients in tissue and suggests that the difficulties in measuring these parameters can be overcome in pursuit of more robust results.

The formulation used in Group 4 contained acetate-esterified XCs and a series of lipids and surfactants that help keep these carotenoid derivatives solubilized in the capsule, without forming microcrystals. These pre-solubilized XCs would be ready for micelle formation in the digestive system for absorption in the intestinal mucosa. On the other hand, free carotenoids form crystals and have to be solubilized by the digestive system prior to incorporation into micelles. This advantage of pre-solubilized acetate-esterified XCs could explain the greater efficiency of Zd and MZd in increasing serum Z and MZ levels when compared to the microcrystalline form of these carotenoids. Therefore, it is striking that this theoretical superiority of acetate-esterified XCs over microcrystals is appreciated for Zd and MZd, but not for Ld. Multiple mechanisms may be preventing Ld from facilitating an increased absorption. For example, L contains an c-ring that is oriented differently from the β-ring of Z and MZ, which seems to affect the position that this XC occupies in lipid membranes. Ld, with an acetate group added to the c-ring, could be positioned less favorably than Zd and MZd in nascent micelles, which could limit its processing by CEL and subsequent contact with the scavenger receptor class B type 1 (SRB-1) for internalization in the intestinal cells. On the other hand, to explain the different behavior of Ld, an alternative hypothesis is provided—L microcrystals could be sufficiently processed in the digestive tract, thus efficiently yielding soluble free L for micelle formation. In this way, Ld would not offer any advantage over L microcrystals, unlike what has been seen with Zd and MZd. It would be necessary to understand the physicochemical behavior of the crystalline form of these xanthophylls in the digestive system to test this hypothesis.

This study describes the behavior of a diacetate formulation in a solubilizate prepared for micellarization with the macular carotenoids compared with crystalline formulations. This study was a double-blind placebo-controlled trial providing high quality of evidence to the field of nutrition and nutritional supplements assessed in a multidisciplinary approach. Our findings provide additional evidence to XC bioavailability. The improved response to Zd and MZd is timely given recent work suggesting that Z and MZ are preferentially accumulated in the human retina over L. However, the importance of the three carotenoids, including L, which collectively contribute to the formation of MP, is acknowledged.

One limitation of the present study is that in order to compare multiple interventions, the sample size in each group had to be reduced. However, using a multiple-arm RCT design overcame sample limitations. Other studies report change-over-time in serum and tissue over longer periods of time, and these reports suggest that sustained supplementation with carotenoids is required to achieve maximal improvements in MPOV and functional outcomes. Even though a longer study is likely to have shown a greater improvement in MPOV, significant improvements in MPOV (for all groups) and skin carotenoid score (for Groups 1 and 4) in this 6-month intervention are generated compared to the placebo. The other challenge faced in clinical studies like this is compliance. Even though RCT guidelines were complied with by counting tablets, XC concentrations in serum were additionally measured at 3 months as an additional compliance marker. Finally, the participants in the current study were healthy Irish adults without known established diseases. Thus, it is not known whether the results would be similar in other ethnic groups, diseases, or children, though it is speculated that the same biologic mechanisms are operative.

### Conclusions

In conclusion, Z and MZ diacetates in a micromicelle-precursor formulation presented an increased bioavailability, most likely due to improved micellarization and absorption efficiency. This formulation is an advance in technology that enhances the bioavailability of Z and MZ when compared to traditional carotenoid supplements.

### Example 2

A micelle-free composition comprising meso-zeaxanthin diacetate in accordance with the current technology, a comparative composition comprising crystalline (3R,3′R)-zeaxanthin, and a placebo (sunflower oil) were provided. The compositions are administered to a subject as tablets.

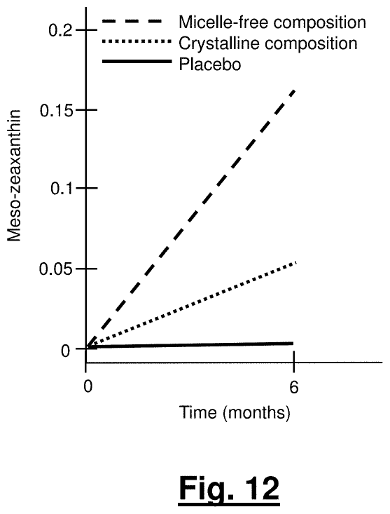

Results are shown in FIG. 12. It can be seen that the micelle-free composition remains bioavailable at higher levels than the placebo and the comparative crystalline composition after 6 months.

A micelle-free composition comprising (3R,3′R,6R)-lutein diacetate, a comparative composition comprising crystalline (3R,3′R,6R)-lutein, and a placebo (sunflower oil) are also provided. The compositions are administered to a subject as tablets.

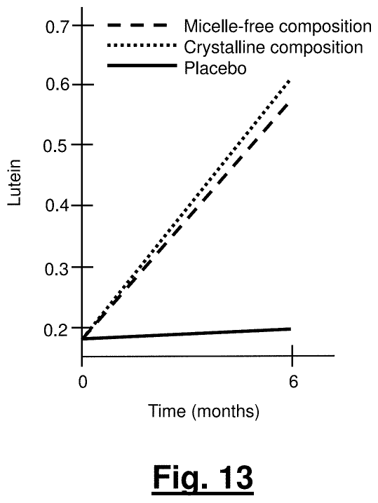

Results are shown in FIG. 13. It can be seen that both the micelle-free composition and the comparative crystalline example remain bioavailable at higher levels than the placebo after six months. However, the levels of (3R,3′R,6R)-lutein provided by the micelle-free composition and comparative example are similar.

This example unexpectedly and surprisingly demonstrates that more of the micelle-free composition comprising meso-zeaxanthin diacetate in accordance with the current technology becomes bioavailable as compared to a crystalline composition comprising meso-zeaxanthin.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A composition comprising:
a xanthophyll carotenoid diacetate;
a transition metal salt; and
phospholipids,
wherein the composition does not comprise micelles, and
wherein the composition is not an emulsion.

2. The composition according to claim 1, wherein the phospholipids are selected from the group consisting of phosphatidylcholines, lysophosphatidylcholines, phosphatidic acids, phosphatidylethanolamines, phosphatidylglycerols, phosphatidylserines, phosphoinositides, phosphosphingolipids, and combinations thereof.

3. The composition according to claim 1, wherein the xanthophyll carotenoid diacetate comprises meso-zeaxanthin diacetate.

4. The composition according to claim 3, further comprising:
(3R,3′R)-zeaxanthin diacetate, (3R,3′R,6R)-lutein diacetate, or a combination thereof.

5. The composition according to claim 1, further comprising:
(3R,3′R,6R)-lutein, (3R,3′R)-zeaxanthin, meso-zeaxanthin, esters thereof, and combinations thereof.

6. The composition according to claim 1, wherein the composition is configured such that micelles encapsulating the xanthophyll carotenoid in free form are formed in a digestive tract of a subject after the composition is orally administered to the subject.

7. The composition according to claim 1, further comprising:
an antioxidant.

8. The composition according to claim 1, wherein the transition metal salt comprises zinc oxide, cupric oxide, cuprous oxide, or combinations thereof.

9. A soft gel capsule comprising the composition according to claim 1.

10. A method of supporting good eye health in a subject in need thereof, the method comprising:
administering a safe and effective amount of a carotenoid composition to the subject, the carotenoid composition comprising:
a xanthophyll carotenoid diacetate;
a transition metal salt; and
phospholipids,
wherein the carotenoid composition does not comprise micelles,
wherein the carotenoid composition is not an emulsion, and
wherein micelles encapsulating the xanthophyll carotenoid in free form are formed from the phospholipids within the digestive tract of the subject.

11. The method according to claim 10, wherein the subject is a human or non-human mammal having below normal levels of macular pigments or at risk of developing age-related macular degeneration (AMD).

12. The method according to claim 10, wherein the xanthophyll carotenoid diacetate is meso-zeaxanthin diacetate, and wherein the carotenoid composition is a gel capsule comprising greater than or equal to about 1% (w/w) to less than or equal to about 30% (w/w) of the meso-zeaxanthin diacetate.

13. The method according to claim 12, wherein the carotenoid composition further comprises (3R,3′R,6R)-lutein and (3R,3′R)-zeaxanthin, the (3R,3′R,6R)-lutein and (3R,3′R)-zeaxanthin optionally being in diacetate forms, and wherein the meso-zeaxanthin diacetate and the (3R,3′R,6R)-lutein are provided in a meso-zeaxanthin diacetate:(3R,3′R,6R)-lutein ratio of from about 1:10 to about 10:1 and the meso-zeaxanthin diacetate and the (3R,3′R)-zeaxanthin are provided in a meso-zeaxanthin diacetate:(3R,3′R)-zeaxanthin ratio of from about 1:1 to about 20:1.

14. The method according to claim 12, wherein the carotenoid composition comprises the meso-zeaxanthin diacetate, (3R,3′R,6R)-lutein, and the (3R,3′R)-zeaxanthin in a meso-zeaxanthin diacetate:(3R,3′R,6R)-lutein:(3R,3′R)-zeaxanthin ratio of about 10:10:2.

15. The method according to claim 10, wherein the transition metal salt comprises at least one of zinc or copper, and wherein the carotenoid composition further comprises sunflower seed oil and at least one of vitamin C or vitamin E.

16. A method of improving the bioavailability of meso-zeaxanthin in a subject, the method comprising:
converting meso-zeaxanthin diacetate into meso-zeaxanthin in free form in the digestive tract of the subject; and
forming micelles within the digestive tract of the subject, the micelles comprising a monolayer of phospholipids encapsulating the meso-zeaxanthin in free form,
wherein more of the meso-zeaxanthin in free form remains biologically available within the blood stream of the subject than in a corresponding meso-zeaxanthin when administered to the subject in crystalline form.

17. The method according to claim 16, wherein the forming micelles within the digestive tract of the subject is a result of administering a safe and effective amount of a carotenoid composition to the subject, the carotenoid composition comprising the phospholipids, the meso-zeaxanthin diacetate, and a transition metal salt, wherein the carotenoid composition is not an emulsion and does not comprise micelles when administered.

18. The method according to claim 17, wherein the carotenoid composition does not comprise gluten.

19. The method according to claim 17, wherein the carotenoid composition further comprises meso-zeaxanthin, (3R,3′R)-zeaxanthin diacetate, (3R,3′R)-zeaxanthin, (3R,3′R,6R)-lutein diacetate, (3R,3′R,6R)-lutein, or combinations thereof.

20. The method according to claim 16, wherein the subject is a human or non-human mammal desiring to maintain or improve macular pigment levels.

21. The method according to claim 16, wherein the subject is a human or non-human mammal having or at risk of developing age-related macular degeneration (AMD).
